(19) United States
(12) Patent Application Publication
(10) Pub. No.: US 2006/0270961 A1
(43) Pub. Date: Nov. 30, 2006

(54) Sample management unit

(75) Inventors: Mark P. Costa, Milton (CA); Davis A.R. Kanbergs, Milton (CA); Taras Worona, Etobicoke (CA); David G. Matsuura, Encinitas, CA (US); Philip J. Simpson, Escondido, CA (US)

Correspondence Address: BUCHANAN, INGERSOLL & ROONEY PC, POST OFFICE BOX 1404, ALEXANDRIA, VA, 22313-1404, US

(73) Assignee: VASOGEN IRELAND LIMITED, Shannon (IE)

(21) Appl. No.: 11/434,217

(22) Filed: May 16, 2006

Related U.S. Application Data
(60) Provisional application No. 60/683,333, filed on May 19, 2005.

(51) Int. Cl. A61M 37/00

(52) U.S. Cl. 604/6.15

(57) ABSTRACT

A vessel having a chamber for receiving an untreated material sample, a chamber inlet for releasably mounting a first syringe and establish a dedicated first fluid coupling therewith to dispense an untreated material sample to the chamber; a chamber outlet for releasably mounting a second syringe and establish a dedicated second fluid coupling with the chamber outlet to dispense a treated material sample following treatment to the second syringe; a gas inlet port coupled to an inlet conduit for carrying at least one gas into the chamber; a gas outlet port coupled to an outlet conduit for carrying at least one gas from the chamber, and a temperature sensor.

### CROSS-REFERENCE TO RELATED APPLICATIONS

This application claims the benefit of priority to U.S. Provisional Application Ser. No. 60/683,333, filed May 19, 2005.

### BACKGROUND OF THE INVENTION

1. Field of the Invention

The present invention relates to a process and apparatus for treating mammalian blood.

2. Description of the Prior Art

Various treatments have been proposed for the treatment of mammalian blood ex vivo to condition the blood in some way before injecting the blood into a patient. Some procedures take blood from a patient, condition the blood, and then return the blood to the same patient continuously. These procedures contrast with procedures which require that the blood be taken from the patient to be treated as a batch and then returned to the patient. In batch processes there is the possibility that the blood will be given to the wrong patient as well as the dangers inherent in transferring blood from one location to another. Also, batch treatments are potentially hazardous because of the risk of blood contamination during the process of conditioning the blood and also because of the potential for infecting the operator accidentally.

It is an object of the present invention to mitigate or obviate at least one of the above-mentioned disadvantages.

### SUMMARY OF THE INVENTION

In one of its aspects, the present invention provides a vessel for the treatment of a material sample, the vessel having:
a top portion, a bottom portion, and a rigid walled portion therebetween;
a cover portion sealing received by a vessel opening adjacent to the top portion to define a treatment cavity;
the cover portion having a plurality of ports in fluid communication with the treatment cavity for ingress or egress of at least one fluid; and
a temperature sensor for determining the temperature of the at least one fluid in the treatment cavity.

In another of its aspects, the present invention provides a vessel for use in a medical treatment system, the vessel having:
a material sample processing chamber having:
a chamber inlet for releasably mounting a first syringe and establish a dedicated first fluid coupling therewith to dispense an unprocessed material sample to the material sample processing chamber;
a chamber outlet for releasably mounting a second syringe and establish a dedicated second fluid coupling with the chamber outlet to dispense a treated material sample following processing to the second syringe;
a gas inlet port coupled to an inlet conduit for carrying at least one gas into the material sample processing chamber;
a gas outlet port coupled to an outlet conduit for carrying at least one gas from the material sample processing chamber; and
a sensor to detecting the ambient temperature of the material sample, the sensor being coupled to a controller for regulating the ambient temperature via a heat source.

In another of its aspects, there is provided a vessel for treating a material sample device for treating a material sample, the vessel having a body comprising:
a proximal end and a distal end;
a rigid portion extending between the proximal end and the rigid end to define a material sample treatment chamber;
a chamber inlet port for releasably mounting a first syringe and establish a dedicated first fluid coupling therewith to dispense an untreated material sample to the material sample treatment chamber near the distal end;
a chamber outlet port for releasably mounting a second syringe and establish a dedicated second fluid coupling with the chamber outlet to dispense a treated material sample following treatment to the second syringe from near the distal end;
a gas inlet port coupled to an inlet conduit for carrying at least one gas into the material sample treatment chamber to cause the at least one gas to bubble the material sample;
a gas outlet port coupled to an outlet conduit for carrying at least one gas from the material sample treatment chamber to remove the at least one gas from the chamber during the treatment and/or after the treatment;
a sensor to detecting the ambient temperature of the material sample, the sensor being coupled to a controller for regulating the ambient temperature via a heat source; and
wherein the chamber inlet port, chamber outlet port, gas inlet port, gas outlet port are located adjacent to the proximal end, and the material sample is contained adjacent the distal end.

In yet another of its aspects, the present invention provides cover for use with a vessel with a top portion, the vessel having a top portion, a bottom portion, and a rigid walled portion therebetween, the vessel having a vessel opening near the top portion to receive the cover to define a enclosed volume and the bottom portion for receiving at least one fluid, the vessel opening having a circumferential flange, the cover comprising:
a top cap with a plurality of ports for coupling with other devices to deliver the at least one fluid into the enclosed volume or to remove the at least one fluid from the enclosed volume, and for receiving a temperature sensor for determining the temperature of the at least one fluid in the enclosed volume;
a manifold abutting the top cap, the manifold having fluid passages corresponding to the plurality of ports and aligned therewith to effect fluid communication with the enclosed volume, the manifold including at least one conduit sealingly interfaced with at least one of the fluid passages at one end, and the other end of the at least one conduit being disposed adjacent to the at least one fluid in the bottom portion;
a cap lock ring resting on the top cap, the cap lock ring including a raised peripheral edge to define a fluid spill reservoir should any fluid spill if the seal between any one of the plurality of ports and the passages fails, the cap lock ring cooperating with a complementary ring abutting the circumferential flange to secure the top cap and manifold to the vessel; and
whereby the rigid walled portion, top portion, bottom portion, and the cover form a fluid sealed vessel.

In yet another of its aspects, the present invention provides a disposable flask assembly conditioning mammalian blood, the flask assembly comprising:
a flask in the form of an envelope defining a substantially enclosed volume, and including a top and a bottom;
the top having an access opening, and a connector assembly is coupled to the top of the flask.
a temperature probe extends from the connector assembly, through the access opening and has a top end and a leading end,
a first conduit sealed in the access opening for transporting a blood sample to the bottom of the flask,
a second conduit for sealed in the access opening transporting the conditioned blood sample from the bottom of the flask out of the flask,
and a gas inlet conduit for feeding gas into the flask to condition the blood sample when a blood sample is in the flask,
a gas outlet conduit for delivering gas out of the flask following the conditioning;
the connector assembly comprising:
   a platform having a first port coupled to the first conduit, the first port having a first connector for coupling a first device thereto to supply the blood,
   a second port coupled to the second conduit having a second connector for coupling a second device thereto to receive the conditioned blood;
   a gas inlet port coupled to the gas inlet conduit for engaging a gas supply system for conditioning the blood; and
whereby the platform includes a raised peripheral edge to define a blood spill reservoir should any blood spill if any of the seals with any of the conduits fails.

In yet another of its aspects, the present invention provides a vessel for processing a material sample, the vessel having:
a material sample processing chamber having:
a chamber inlet for releasably mounting a first syringe and establish a dedicated first fluid coupling therewith to dispense an unprocessed material sample to the material sample processing chamber;
a chamber outlet for releasably mounting a second syringe and establish a dedicated second fluid coupling with the chamber outlet to dispense a treated material sample following processing to the second syringe;
a gas inlet port coupled to an inlet conduit for carrying at least one gas into the material sample processing chamber;
a gas outlet port coupled to an outlet conduit for carrying at least one gas from the material sample processing chamber; and
a sensor to detecting the ambient temperature of the material sample, the sensor being coupled to a controller for regulating the ambient temperature via a heat source.

In yet another of its aspects, the present invention provides a sample management device for use in a medical treatment system, the device comprising:
a body having:
   a top portion, a bottom portion, and a rigid walled portion therebetween;
   a cover portion sealing received by a body opening adjacent to the top portion to define a treatment cavity;
   a first syringe;
   a second syringe;
   the cover portion having a plurality of ports in fluid communication with the treatment cavity; the first syringe being releasably coupled to at least one of the plurality of ports for supplying an untreated fluid, and the second syringe being releasably coupled to at least one of the plurality of ports for receiving a treated fluid; the plurality of ports including a gas inlet port coupled for carrying at least one gas into the treatment cavity to interface with the untreated sample, and also including a gas outlet port coupled for carrying at least one gas from the treatment cavity; and
a temperature sensor for determining the temperature of the at least one fluid in the treatment cavity.

### BRIEF DESCRIPTION OF THE DRAWINGS

These and other features of the preferred embodiments of the invention will become more apparent in the following detailed description in which reference is made to the appended drawings wherein:

FIG. 1 is a perspective view of a material treatment system;

FIG. 2 is a perspective view of a vessel of material treatment system of FIG. 1;

FIG. 3 is a sectional view of a first syringe shown in FIG. 1, taken along line 3-3′;

FIG. 4 is an exploded view of the vessel of FIG. 2;

FIG. 5 is a perspective view of a top portion of the vessel of FIG. 2;

FIG. 6 is a perspective view of a cover portion of the vessel of FIG. 2;

FIG. 7 is sectional view of the cover portion of the vessel of FIG. 2 taken along line 7-7′;

FIG. 8 is an exploded view of the cover portion;

FIG. 9 is a perspective view of the cover portion with conduits coupled thereto;

FIG. 10 is a partial sectional view of the vessel of FIG. 2 taken along line 10-10′;

FIG. 11 is a sectional view of the second syringe of FIG. 2 taken along line 11-11′;

FIG. 12 is a bottom perspective view of the cover portion with conduits coupled thereto;

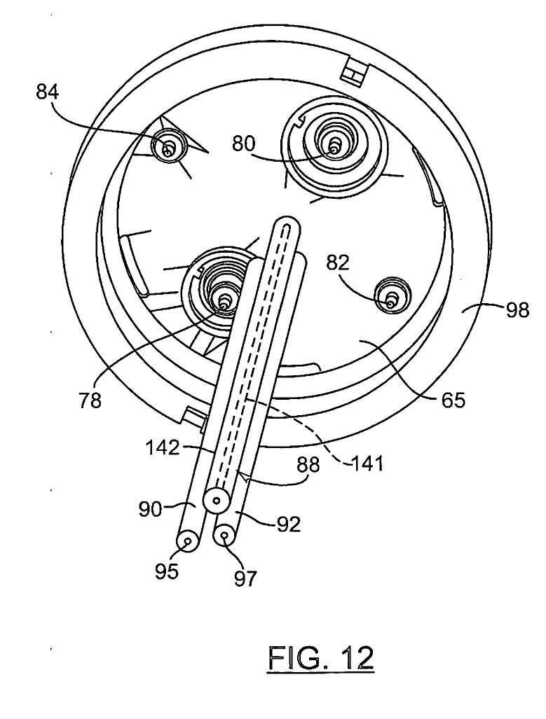

FIG. 13 is perspective view of a cover portion of the vessel in another embodiment; and

FIG. 14 is a sectional view of the cover portion of FIG. 13 taken along line 13-13′.

### DESCRIPTION OF THE PREFERRED EMBODIMENTS

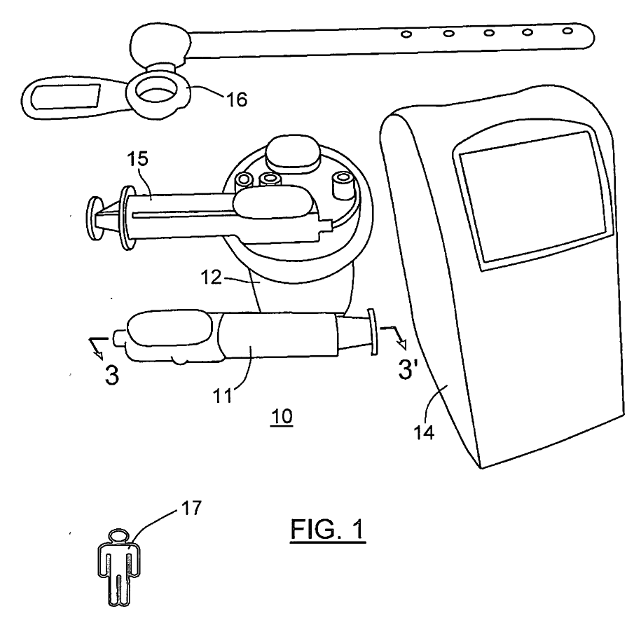

As shown FIG. 1, there is provided a system 10 for the collection, treatment and delivery of a material, such as an autologous blood sample. The system 10 includes a plurality of entities which are used at different stages during the handling of the blood sample, such as, a the blood collection syringe or first syringe 11, a blood sample management unit 12, a blood treatment unit 14, a blood delivery syringe or second syringe 15, and a wristband 16. The first syringe 11 is used to collect an untreated blood sample from an originating patient 17. Following collection of the untreated blood sample, the blood collection syringe 11 is coupled to the sample management unit with the blood delivery syringe 15 already mounted thereon, and the sample management unit is introduced into the blood treatment unit, in which the untreated blood sample is subjected to one or more stressors, such as ozone or ozone/gas mixture, ultra-violet (UV) light and infra-red (IR) energy.

Following treatment, the treated blood sample is extracted into the second syringe 15, from which the treated blood sample is administered to the originating patient 17. At one or more critical stages, the system 10 provides for a verification check, aimed at reducing the possibility of error, to ensure that the correct blood sample is returned to the correct originating patient 17. This is done by matching the blood sample, either in its treated or untreated form or both, with the originating patient 17. Typically, the wristband 16, the first syringe 11, the sample management unit 12, the second syringe 15, include identification data associated with the originating patient, the data may include indicia, or may be machine-readable via optical or electro/magnetic means.

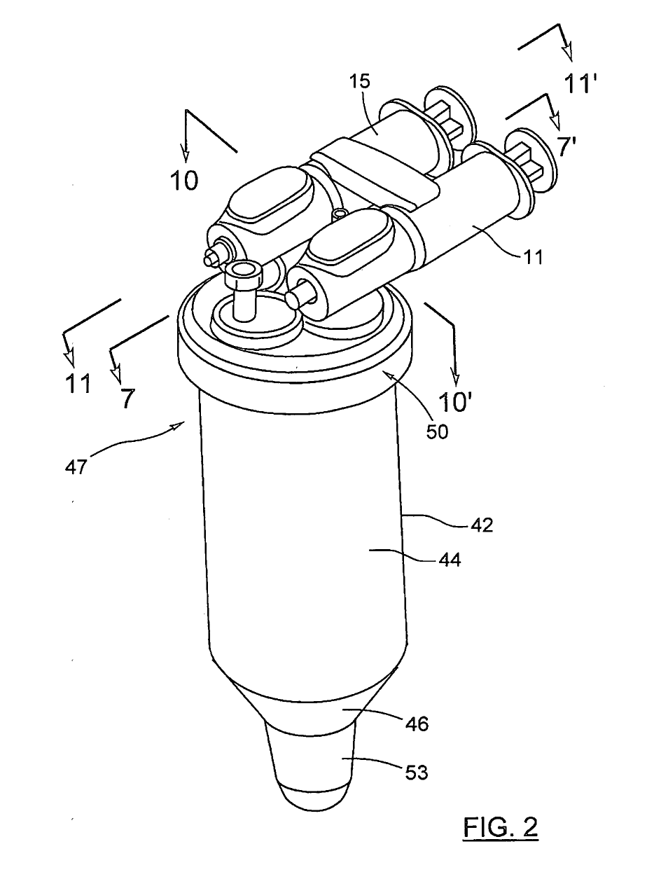

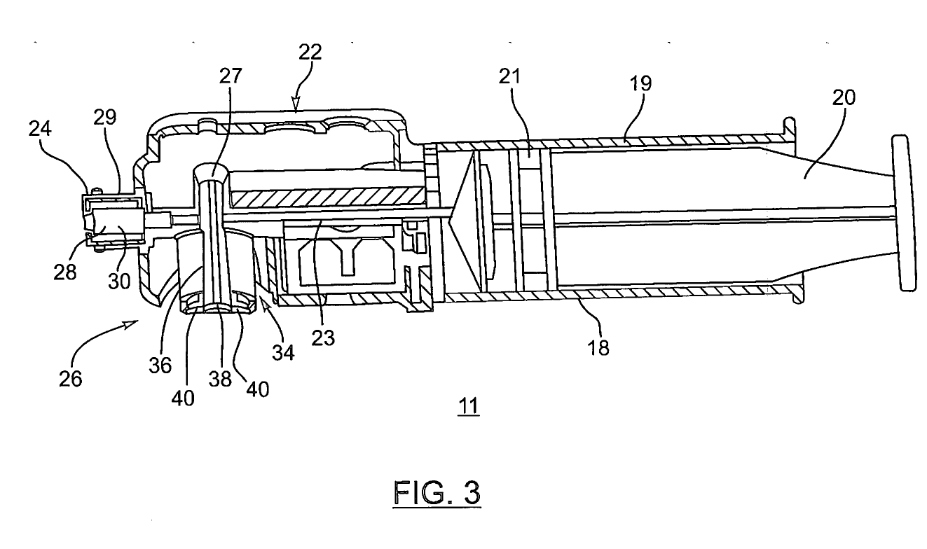

As shown in FIGS. 2 and 3, the first syringe 11 has a first body portion 18 which provides a cylindrical cavity 19 which in cooperation with a syringe plunger 20 forms a sample receiving chamber 21. The first syringe 11 includes a first channel portion 22 with a channel 23 in communication with the sample receiving chamber 21, and a first syringe inlet port 24 for ingress of the untreated blood sample. The first channel portion 22 also includes a first syringe outlet port 26 for dispensing the untreated blood sample therefrom to the sample management unit 12. The first syringe outlet port 26 includes a channel 27 in communication with the channel 23 in a cruciform arrangement.

The first syringe inlet port 24 is provided with a first syringe inlet valve means 28 for controlling the flow of blood through the first syringe inlet 24. In this case, the first inlet valve means 28 includes a housing 29 containing a valve 30 arranged to be opened by a complementary valve member located on an external device (not shown). The external device may be a blood collection unit, such as a “butterfly” needle or a sodium citrate bag, and so forth. Extending outwardly from the first syringe outlet port 26 is a pair of bayonet pins 34 for coupling the first syringe 11 to the sample management unit 12. Included within the channel 23 of the first syringe 11 is a valve element 36 biased to a closed position against a valve seat 38 on an end cap 40 which forms the outer end of the first syringe outlet port 26.

The first syringe 11, second syringe 15, and the sample management unit 12, may include circuitry for transmitting and receiving data related to the syringe and/or its contents, or a patient, such as identification data, SKU, serial no., manufacturing date, expiry date, fluid data, health facility data, health practitioner data, medication data, authentication data, and so forth. The data, or portions of the data, may also be secured via encryption algorithms and schemes, to ensure data integrity and/or authenticity of the entity. The circuitry may include, but is not limited to, a transmitter, a receiver, logic means or processor, a memory for data storage, a timing circuit, an antenna, a power source, input/out devices such as a display, an LED, a speaker, and a switch. As an example, the circuitry may include a radio identification (RFID) integrated circuit associated with an antenna or an RFID tag.

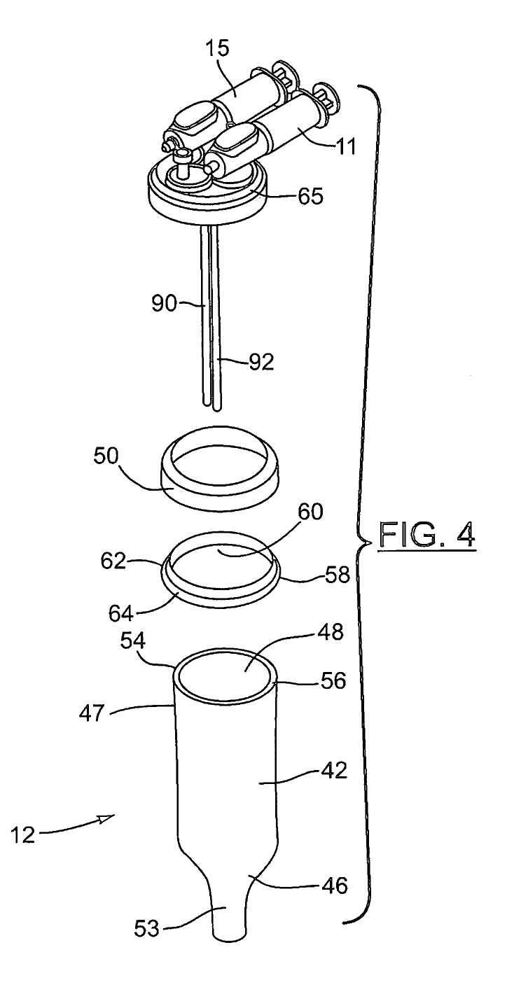

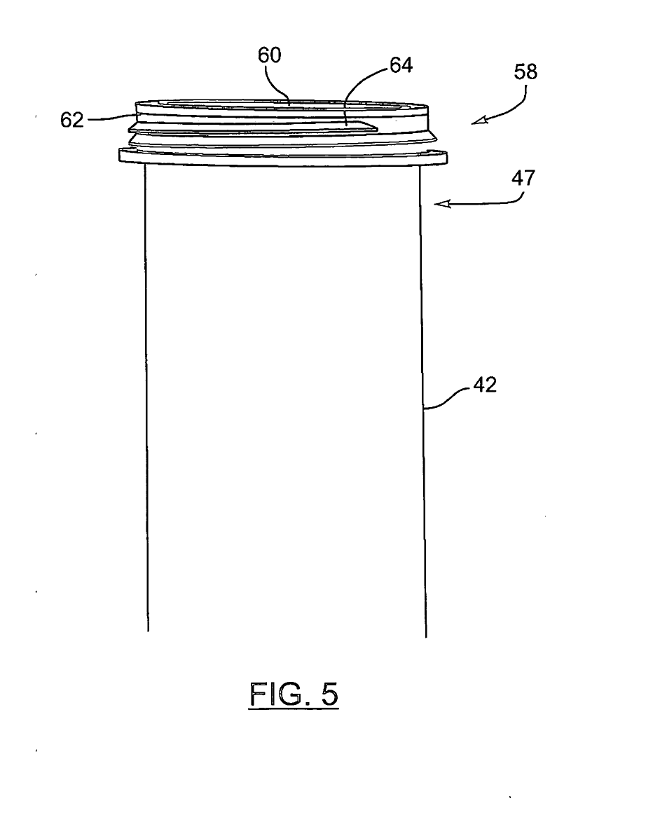

Below is a description of the treatment portion of the blood treatment process involving the use of the sample management unit 12 in the blood treatment unit 14. As shown in FIG. 2 to 6, the sample management unit 12 is a vessel 42 including a rigid walled portion 44 with a bottom portion 46, a top portion 47 with a vessel opening 48 which receives a cover portion 50, to define a treatment chamber or treatment cavity 52. The vessel 42 includes an inner wall 49 and outer wall 51, and the bottom portion 46 has a bowl 53 to receive the blood sample. The vessel opening 48 includes a rim 54 with an annular flange 56 extending outwardly from the rigid walled portion's 44 outer wall 57. A threaded ring 58 abutting the annular flange 56 has one side 60 which engages the outer wall near the top portion 47, and the other side 62 bears threads 64 to receive a complementary threads, as shown in FIGS. 4 and 5. The cover portion 50 includes a top cap 65 with a gas inlet port 66 for delivery of ozone to treat a blood sample, a gas outlet port 68 for the discharge of the ozone, and other gases from the treatment cavity 52. Also included in the top cap 65 is a chamber inlet 70 to form a dedicated first fluid coupling with the first syringe outlet port 26, such that the untreated blood sample may be dispensed into the treatment cavity 52, a chamber outlet 72 to form a dedicated second fluid coupling with the second syringe 15.

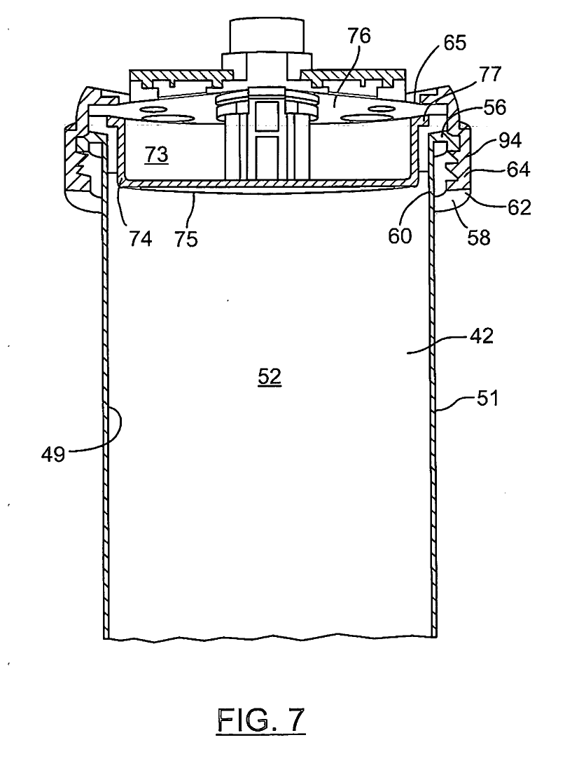

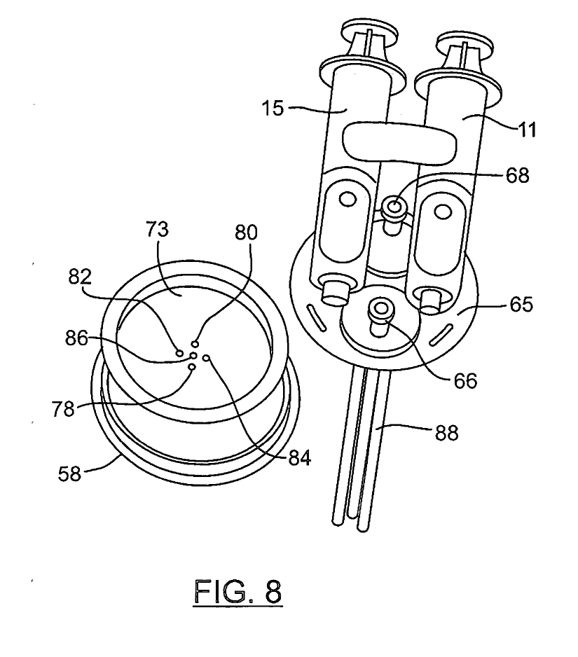

As shown in FIG. 7, the cover portion 50 further comprises a manifold 73 received in the vessel opening 48. The manifold 73 includes a side portion 76 sealingly engaging the vessel's 42 inner wall 70, a manifold bottom portion 75 facing the treatment cavity 52 and a manifold top portion 76 with a lip 77 abutting the annular flange 56, such that the threaded ring 58 and the lip 77 flank the annular flange 56. The manifold 73 is interfaced with the top cap 65, the manifold top portion 76 includes fluid passages 78, 80, 82, 84 to deliver the blood sample and gases in and out of the treatment cavity 52, as shown in FIG. 8. The passages 78, 80, 82, 84 correspond to the gas inlet port 66 or gas inlet needle port, gas outlet port 68 or gas exhaust needle port, chamber inlet port 70, chamber outlet port 72, respectively.

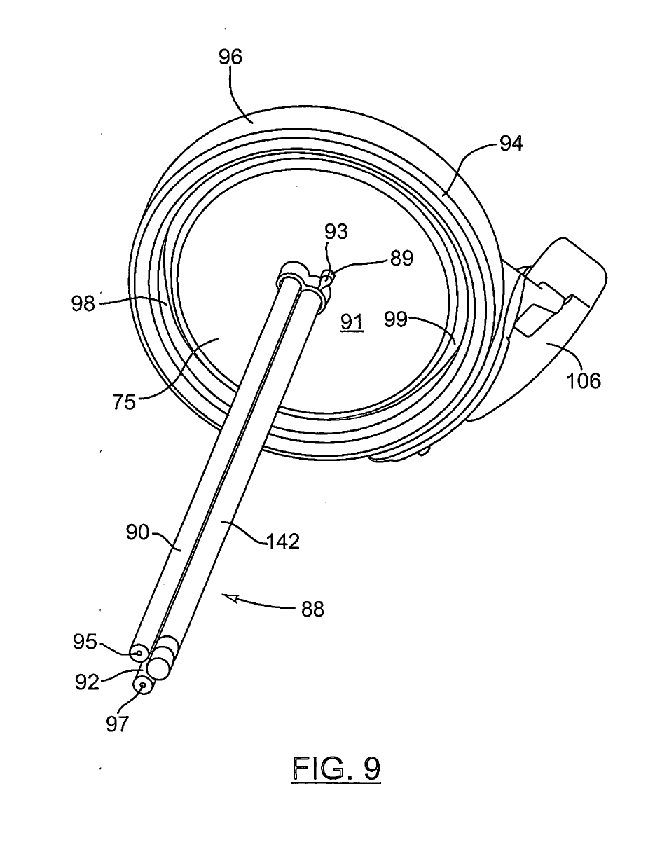

As shown in FIG. 9, the passages 78, 80, 82 and 84 extend from the manifold top portion 76 to the manifold bottom portion 75. Also included in the manifold 73 is a passage 86 for receiving a temperature sensor, such as a thermistor assembly 88, in close proximity with the bottom portion of the bowl 53, and in contact with the blood sample. The passage 82 is coupled to a short conduit 89 extending from the manifold bottom portion 75, the conduit 89 introduces untreated blood into the vessel 42. The treated blood sample is delivered to the second syringe. 15 via a conduit 90 coupled to the passage 84. Coupled to the passage 78 is a conduit 92 for delivering the ozone gas or gas mixtureto the untreated blood sample in the bowl 53. The passage 80 terminates on the manifold bottom portion 75 to define an aperture 91 which interfaces with the interior of the vessel 42, and serves to purge gases from the treatment cavity 52 via the port 68. The conduit 90 and/or conduit 92 may be a rigid tube in a sealing condition with the manifold 73, while the conduit 89 is co-molded with. the manifold 73. The untreated blood sample is forced down via a lumen 93 in the conduit 89. The conduit 89 is adjacent to the thermistor assembly 88, and terminates at an angle such that the untreated blood sample is caused to travel down the exterior wall of the thermistor assembly 88. This configuration helps to minimise blood loss due to residual blood in the lumen 91. The conduit 90 includes a lumen 95 with one end in fluid communication with the passage, while the other end of the lumen 95 is in contact with the blood pool in the bowl 53 to ensure the maximum removal of treated blood from the bowl 53. The conduit 92 includes a lumen 97 to supply the ozone gas or gas mixture to the untreated blood.

The gas ports 66, 68 include a filter, such as a hydrophobic filter to help prevent the ingress and egress of biological components or debris from entering, or leaving, the treatment cavity 50. The hydrophobic filter also helps to prevent clogging of the filter by substantially preventing blood bubbles from accessing the filter. Generally, the gas is exhausted during treatment, as such, the gas outlet passage 80 is disposed close to the top portion 47 to substantially avoid contact with the blood bubbles present during treatment. Also included is an anti-viral filter media to help prevent bio-organisms, bacteria and viri from entering, or leaving, the treatment cavity 50. For example, the anti-viral filter media is a 0.2 micron filter from Gore, USA, model GORE MMT 316.

As shown in FIG. 9, there is a cap lock ring 94 which secures the top cap 65 and the manifold 73, in cooperation with the threaded ring 58. The cap lock ring 94 includes a cap lock ring portion 96 with a raised peripheral circumferential edge abutting the top cap 65 and a cap lock ring threaded portion 98 with complementary threads 99 which received the threads 60 of the threaded ring 58. The cap lock ring 94 envelops the edges of the annular flange 56, the lip 77 and the top cap 65, and thus the annular flange 56, the lip 77 and top cap 65 are sandwiched between the cap lock ring portion 96 and the threaded ring 58. Therefore, a cap lock ring portion 96 is therefore elevated above the top cap 65, when the sample management unit 12 is position in an upright manner, as illustrated. Following assembly of the vessel 42 components, the vessel 42 is liquid sealed by bonding or welding together the cap lock ring 94, the top cap 65, the manifold 73, the threaded ring 58, annular flange 56, and the top portion 47. The elevation of the cap lock ring portion 96 above the top cap 65 defines a blood spill reservoir zone should any blood spill if the seal between the first syringe 11 and the chamber inlet port 70 fails. Thus, the blood collects on the top cap 65 and is contained there by the elevated cap lock ring portion 96, and any blood spills into the blood treatment unit 14 are substantially eliminated.

In more detail, the chamber inlet port 70 includes a female collar portion 100 with a pair of helically oriented passages or grooves 102 extending through or in its wall to receive the pair of corresponding bayonet pins 34 of the first syringe outlet port 26. The base of the chamber inlet port 70 is a valve-actuating element 104 which abuts the valve element 36 when the first syringe 11 is received by the chamber inlet port 70. In operation, the bayonet pins 34 travel along the helical passages 102 and the valve-actuating element 104 displaces the valve element 36 from its closed position against the valve seat 38 to open the first fluid coupling. Once fully engaged with chamber inlet port 70, the first syringe 11 is supported in place by a saddle member 106, which minimizes motion of the first syringe 11 about the chamber inlet 70.

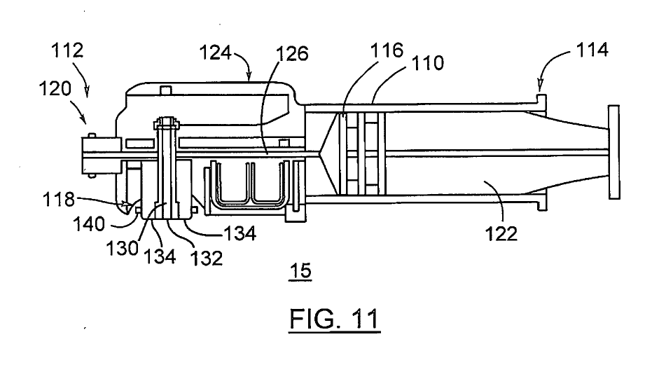

Correspondingly, the chamber outlet port 72 receives the second syringe 15. As shown in FIG. 11, the second syringe 15 has a second body portion 110 having a proximal end 112 and a distal end 114, with a second fluid receiving chamber 116 in fluid communication with a second inlet port 118 and a second outlet port 120 coupled thereto near the proximal end 112. A plunger 122 is slidably disposed in the second fluid receiving chamber 116 at the distal end 114, the plunger 122 serves to draw fluid into the second fluid receiving chamber 116 and urge the fluid therefrom. The second syringe 15 also includes a second channel portion 124 with a channel 126 in communication with the second chamber 116 and the second outlet port 120, and a channel 128 in communication with the second inlet port 118 and the second chamber 116 via a portion of the channel 126. A valve element 130 is located in the channel 128 and biased to a closed position against a valve seat 132 on an end cap 134 forming the outer end of the second syringe inlet port 118. The second syringe outlet port 120 includes a releasable lock means which allows the treated blood to be administered to the patient only when the identity of the treated blood sample matches with the originating patient. The chamber outlet port 72 is so dimensioned to only receive the second syringe 15, and the chamber inlet port 70 only receives the first syringe 11, thus preventing errors in the treatment procedure and reducing waste from failed or unfulfilled treatments.

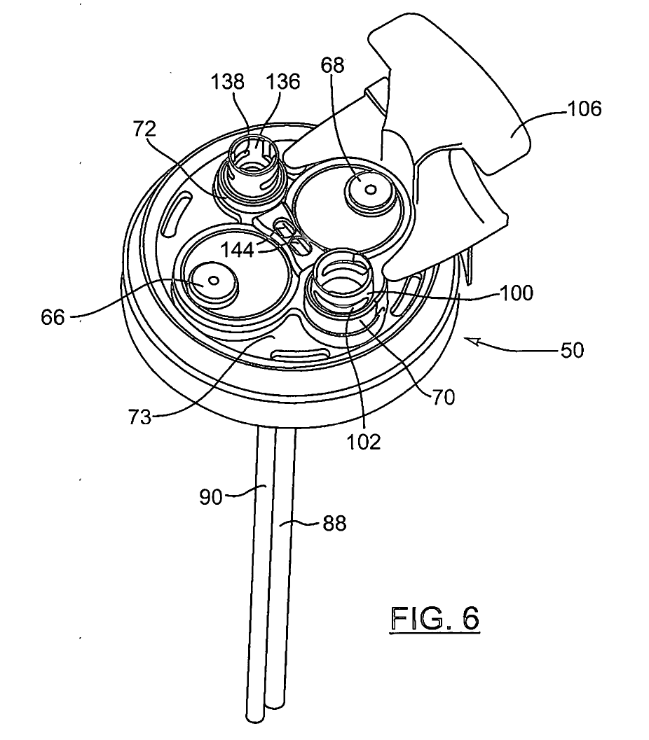

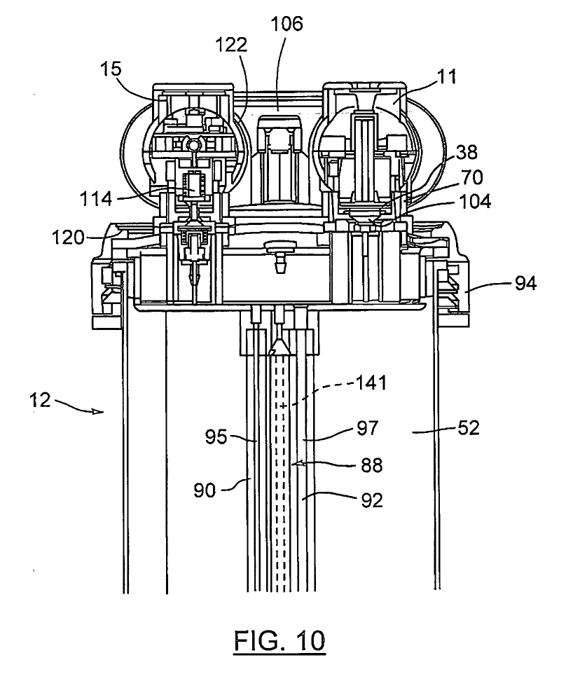

As shown in FIGS. 6 and 10, the chamber outlet port 72 has a female collar portion 136 with a pair of helically oriented passages or grooves 138 extending through or in its wall to engage a corresponding one or more pins 140 extending outwardly from the second syringe inlet port 118. The valve element 130 is also aligned for abutment with a valve actuating element 120 which is positioned in the chamber outlet port 72. The valve actuating element 120 is thus operable to displace the valve element 130 from its closed position against the valve seat 132 to open the second fluid coupling. The saddle member 106 also supports the second syringe 15 when it is in a fully engaged position with chamber outlet port 72, such that the first syringe 11 and the second syringe 15 are adjacent to each other when positioned on the cover portion 50.

With the first syringe 11 and the second syringel 5 mounted on the cover portion 50, the sample management unit 12 is received by the blood treatment unit 14. The RFID tag on the sample management unit 12 is read by an RFID reader/writer associated with the blood treatment unit 14 to verify authenticity of the sample management unit 12. Also, subsequent to the delivery of the blood sample to the treatment cavity 52, and treatment of same, the RFID tag on the sample management unit 12 receives a disable code from the blood treatment unit 14, thereby preventing the reuse of the sample management unit 12. Alternatively, the RFID tag may be rendered inoperable by an external signal causing a fuse to be blown therein or to destroy the antenna or receiver/transmitter.

In the course of treatment, untreated blood sample in the treatment cavity 52 is subjected to one or more stressors, such as ozone or ozone/oxygen mixture, ultra-violet (UV) light (A, B and C radiation) and infrared (IR) energy, via the walled portion 46 and the bowl 53. As such, walled portion 46 and the bowl 53 are made from appropriate materials capable of transmitting such radiation, such as low-density polyethylene (LDPE) containing a small amount (about 5%) of ethylene vinyl acetate. The thermistor assembly 88 includes a thermistor 141 to sense the blood temperature within the blood pool and the cavity 52 ambient temperature during the treatment process. The thermistor 141 is housed in a thermistor down tube 142 made from biocompatible material with substantially high thermal conductivity. The tube 142 also includes a sufficiently large surface area and minimal thickness to effectively transfer heat to the thennistor 141 in a relatively short time period, as shown in FIG. 9. As such, the thermistor 141 is coupled to electrical contacts 144 on the top cap 65, and the electrical contacts 144 are in turn coupled to electronic circuitry or logic means of the blood treatment unit. Using an output from the thermistor 141, the electronic circuitry or logic means can determine whether a predetermined blood pool temperature has been reached, otherwise the electronic circuitry or logic means adjusts the infrared heat output. Following the necessary steps of the treatment procedure, such as, comparing the patient wristband label and second syringe label, and ensure they carry identical patient information, the treated blood is administered to the patient.

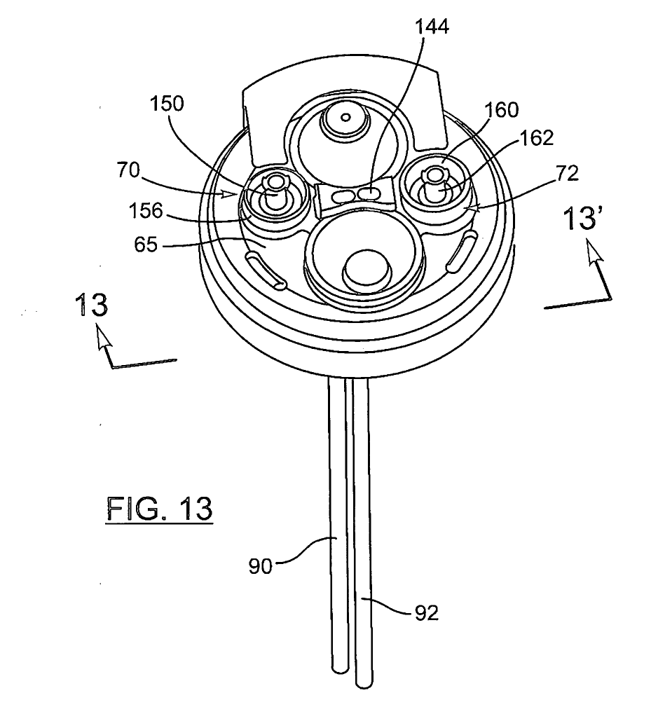

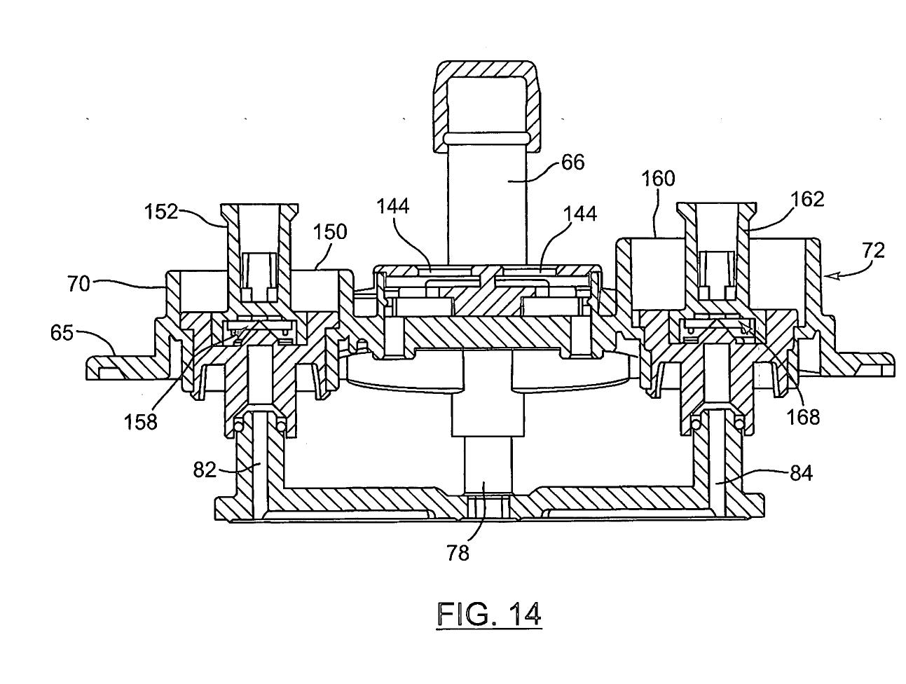

In another embodiment, as shown in FIGS. 13 and 14, the vessel 42 includes a cover portion 149, which includes like elements as found in the cover portion 50, with the exception that the chamber inlet port 70 is a top mount with a Luer connector 150 with a vent cap 152. The Luer connector 150 receives a corresponding Luer connector 154 on a first syringe 156 (not shown), such as a conventional syringe. The syringe 156 is received vertically on the top cap 65. The chamber inlet port 70 includes a valve 158, such as a duckbill valve, which remains closed when there is no blood flow from the first syringe 156, and is opened when the flow rate increases. Thus, the valve remains open when the first syringe 156 is engaged with chamber inlet port 70 while the untreated blood is expressed into the treatment cavity 52, while preventing backflow. The first syringe 156 is subsequently removed following the dispensing of the untreated blood into the treatment cavity. Disengaging the first syringe 154 closes the valve 158 and seals the treatment cavity from the exterior. The vessel 42 is then introduced into the blood treatment unit 14 and in the course of the treatment of the blood sample, the treatment cavity 52 is subjected at least one stressor, such as ozone or ozone/oxygen gas mixture, UV A, B and C radiation, and infrared radiation.

Correspondingly, the chamber outlet port 72 is a top mount with a Luer connector 160 with a vent cap 162. The Luer connector 160 receives a corresponding Luer connector 164 on a second syringe 166 (not shown), such as a conventional syringe. The syringe 166 is also received vertically by the top cap 65. The chamber outlet port 72 includes a valve 168, which is opened when the second syringe 166 is engaged with chamber outlet port 72, to allow the treated blood to be extracted from the treatment cavity 52 into the second syringe 166, after the blood treatment, while preventing backflow. Following the necessary steps of the treatment procedure, such as, comparing the patient wristband label and second syringe label, and ensure they carry identical patient information, the treated blood is administered to the patient.

Even though the description above is in large part focused on the treatment of blood samples, it will be understood that the system 10, its components and alternatives thereof, may be used for samples other than blood samples, such as bone marrow or, lymphatic fluids, semen, ova- fluid mixtures, other bodily fluids or other medical fluids which may or may not be “autologous”, for example fluid mixtures perhaps containing a patient's desired solid sample such as from organs, body cells and cell tissue, skin cells and skin samples, spinal cords. The device 10 may also be used for medical testing where it is important to ensure that test results of a particular test can be delivered to the originating patient 17.

Although the invention has been described with reference to certain specific embodiments, various modifications thereof will be apparent to those skilled in the art without departing from the spirit and scope of the invention as outlined in the claims appended hereto.

The embodiments of the invention in which an exclusive property or privilege is claimed are defined as follows:

1. A vessel for use in a medical treatment system, the vessel having:
a top portion, a bottom portion, and a rigid walled portion therebetween;
a cover portion sealing received by a vessel opening adjacent to the top portion to define a treatment cavity;
the cover portion having a plurality of ports in fluid communication with the treatment cavity for ingress or egress of at least one fluid; and
a temperature sensor for determining the temperature of the at least one fluid in the treatment cavity;
wherein the rigid walled portion, top portion, bottom portion, and cover portion are assembled to form a fluid sealed vessel.

2. The vessel of claim 1 wherein the at least one fluid is a blood sample.

3. The vessel of claim 2 wherein the cover portion includes a gas inlet port, gas outlet port, a treatment cavity inlet port and a treatment cavity outlet port.

4. The vessel of claim 3 wherein the cover portion further includes a top cap with the plurality of ports and a manifold abutting the top cap, the manifold having fluid passages corresponding to the plurality of ports and aligned therewith to effect fluid communication with the treatment cavity.

5. The vessel of claim 4 wherein the cover portion further includes a cap lock ring resting on the top cap to secure the top cap and manifold to the top portion of the vessel

6. The vessel of claim 5 wherein the manifold includes at least one conduit sealingly interfaced with at least one of the plurality of ports at one end, and the other end adjacent to the at least one fluid in the bottom portion.

7. The vessel of claim 6 wherein the cap lock ring includes a raised peripheral edge to define a fluid spill reservoir should any fluid spill if the seal between any one of at least one of the plurality of ports and the at least one conduit fails.

8. The vessel of claim 5 wherein the manifold includes electric contacts for coupling the temperature sensor to the system.

9. A vessel for processing a material sample, the vessel having:
a material sample processing chamber having:
a chamber inlet for releasably mounting a first syringe and establish a dedicated first fluid coupling therewith to dispense an unprocessed material sample to the material sample processing chamber;
a chamber outlet for releasably mounting a second syringe and establish a dedicated second fluid coupling with the chamber outlet to dispense a treated material sample following processing to the second syringe;
a gas inlet port coupled to an inlet conduit for carrying at least one gas into the material sample processing chamber;
a gas outlet port coupled to an outlet conduit for carrying at least one gas from the material sample processing chamber; and
a sensor to detecting the ambient temperature of the material sample, the sensor being coupled to a controller for regulating the ambient temperature via a heat source.

10. A vessel for treating a material sample, the vessel having a body comprising:
a proximal end and a distal end;
a rigid portion extending between the proximal end and the rigid end to define a material sample treatment chamber;
a chamber inlet port for releasably mounting a first syringe and establish a dedicated first fluid coupling therewith to dispense an untreated material sample to the material sample treatment chamber near the distal end;
a chamber outlet port for releasably mounting a second syringe and establish a dedicated second fluid coupling with the chamber outlet to dispense a treated material sample following treatment to the second syringe from near the distal end;
a gas inlet port coupled to an inlet conduit for carrying at least one gas into the material sample treatment chamber to cause the at least one gas to bubble the material sample;
a gas outlet port coupled to an outlet conduit for carrying at least one gas from the material sample treatment chamber to remove the at least one gas from the chamber during the treatment and/or after the treatment;
a sensor to detecting the ambient temperature of the material sample, the sensor being coupled to a controller for regulating the ambient temperature via a heat source; and
wherein the chamber inlet port, chamber outlet port, gas inlet port, gas outlet port are located adjacent to the proximal end, and the material sample is contained adjacent the distal end.

11. A disposable flask assembly for conditioning mammalian blood, the flask assembly comprising:
a flask in the form of an envelope defining a substantially enclosed volume, and including a top and a bottom;
the top having an access opening, and a connector assembly is coupled to the top of the flask.
a temperature probe extends from the connector assembly, through the access opening and has a top end and a leading end,
a first conduit sealed in the access opening for transporting a blood sample to the bottom of the flask,
a second conduit for sealed in the access opening transporting the conditioned blood sample from the bottom of the flask out of the flask,
and a gas inlet conduit for feeding gas into the flask to condition the blood sample when a blood sample is in the flask,
a gas outlet conduit for delivering gas out of the flask following the conditioning;
the connector assembly comprising:
a platform having a first port coupled to the first conduit, the first port having a first connector for coupling a first device thereto to supply the blood,
a second port coupled to the second conduit having a second connector for coupling a second device thereto to receive the conditioned blood;
a gas inlet port coupled to the gas inlet conduit for engaging a gas supply system for conditioning the blood; and
whereby the platform includes a raised peripheral edge to define a blood spill reservoir should any blood spill if any of the seals with any of the conduits fails.

12. The disposable flask assembly of claim 11 wherein the connector assembly includes Luer connectors for coupling to the devices with complementary Luer connectors.

13. The disposable flask assembly of claim 11 wherein the connector assembly includes bayonet locking mechanism for coupling to the devices with complementary bayonet locking mechanism.

14. A cover for use with a vessel, the vessel having a top portion, a bottom portion, and a rigid walled portion therebetween, the vessel having a vessel opening near the top portion to receive the cover to define a enclosed volume and the bottom portion for receiving at least one fluid, the vessel opening having a circumferential flange, the cover comprising:
a top cap with a plurality of ports for coupling with other devices to deliver the at least one fluid into the enclosed volume or to remove the at least one fluid from the enclosed volume, and for receiving a temperature sensor for determining the temperature of the at least one fluid in the enclosed volume;
a manifold abutting the top cap, the manifold having fluid passages corresponding to the plurality of ports and aligned therewith to effect fluid communication with the enclosed volume, the manifold including at least one conduit sealingly interfaced with at least one of the fluid passages at one end, and the other end of the at least one conduit being disposed adjacent to the at least one fluid in the bottom portion;
a cap lock ring resting on the top cap, the cap lock ring including a raised peripheral edge to define a fluid spill reservoir should any fluid spill if the seal between any one of the plurality of ports and the passages fails, the cap lock ring cooperating with a complementary ring abutting the circumferential flange to secure the top cap and manifold to the vessel; and
whereby the rigid walled portion, top portion, bottom portion, and the cover form a fluid sealed vessel.

15. The cover of claim 14 wherein the at least one fluid is a biological fluid material.

16. The cover of claim 14 wherein the at least one fluid is a gaseous material.

17. The cover of claim 15 wherein the vessel is used for treating the biological fluid as part of a medical treatment.

18. The cover of claim 16 wherein the vessel is used for treating the biological fluid as part of a medical treatment.

19. The cover of claim 17 wherein the least one conduit is in contact with the biological fluid in the bottom portion.

20. The cover of claim 18 wherein the least one conduit is in contact with the biological fluid in the bottom portion.

21. The cover of claim 14 wherein the ports include Luer connectors for coupling to the devices having complementary Luer connectors.

22. The cover of claim 14 wherein the ports include a bayonet coupling part for coupling to a complementary bayonet coupling part of the other devices.

23. A sample management device for use in a medical treatment system, the device comprising:
a body having:
a top portion, a bottom portion, and a rigid walled portion therebetween;
a cover portion sealing received by a body opening adjacent to the top portion to define a treatment cavity;
a first syringe;
a second syringe;
the cover portion having a plurality of ports in fluid communication with the treatment cavity; the first syringe being releasably coupled to at least one of the plurality of ports for supplying an untreated fluid, and the second syringe being releasably coupled to at least one of the plurality of ports for receiving a treated fluid; the plurality of ports including a gas inlet port coupled for carrying at least one gas into the treatment cavity to interface with the untreated fluid, and also including a gas outlet port coupled for carrying at least one gas from the treatment cavity;
a temperature sensor for determining the temperature of the at least one fluid in the treatment cavity.

24. The device of claim 23 wherein each of the first syringe, second syringe, device includes an identifier.

25. The device of claim 24 wherein the identifier includes indicia, or is machine- readable optically or electro/magnetically.

26. The device of claim 25 wherein the identifier includes an RFID tag, the RFID tag having a computer readable medium associated with any of the following data related to the first syringe, the second syringe, the device and/or contents therein, patient identification data, SKU, serial no., manufacturing date, expiry date, fluid data, health facility data, health practitioner data, medication data, authentication data, integrity data, encryption data.
